(19) United States
(12) Patent Application Publication
(10) Pub. No.: US 2009/0305011 A1
(43) Pub. Date: Dec. 10, 2009

(54) Ink Jet Ink

(75) Inventors: Ralph McIntosh, Hanau (DE); Gerd Tauber, Seligenstadt (DE); Werner Kalbitz, Rodenbach (DE); Stephan Lüdtke, Gelnhausen (DE); Jutta Zimmermann, Alzenau (DE); Alfons Karl, Grundau (DE); Frank Forster, Schollkrippen (DE)

Correspondence Address: LAW OFFICE OF MICHAEL A. SANZO, LLC, 15400 CALHOUN DR., SUITE 125, ROCKVILLE, MD, 20855, US

(73) Assignee: EVONIK DEGUSSA GMBH, Essen (DE)

(21) Appl. No.: 12/475,517

(22) Filed: May 30, 2009

(30) Foreign Application Priority Data
Jun. 5, 2008 (DE) 10 2008 026 894.1

(51) Int. Cl. B32B 3/10; C07C 63/16; C07C 309/29; C07C 63/20; B32B 29/00

(52) U.S. Cl. 428/211.1; 562/480; 562/74; 428/195.1

(57) ABSTRACT

Ink jet inks comprising a modified pigment, wherein the modified pigment is a pigment having attached organic groups of substituted or unsubstituted 1,2-benzenedicarboxylic acids and/or salts thereof.

### CROSS REFERENCE TO RELATED APPLICATIONS

The present application claims priority to, and the benefit of, application DE 10 2008 026 894.1, filed in Germany on Jun. 5, 2008, the contents of which is hereby incorporated by reference in its entirety.

### FIELD OF THE INVENTION

The invention relates to an ink jet ink and also to its use.

### BACKGROUND OF THE INVENTION

U.S. Pat. No. 5,922,118 and U.S. Pat. No. 5,554,739 disclose modified pigments having aromatic or aliphatic groups.

EP 1 061 106 discloses ink jet inks comprising a colorant having functional groups, of which ester groups

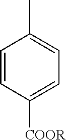

improve colour bleed control and waterfastness and

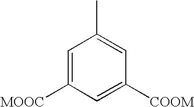

groups improve water dispersibility.

EP 1 061 107 discloses ink jet inks comprising a colorant having functional groups, of which —R₁—COOM and —Ar—CONHR₁COOM improve colour bleed control and waterfastness and

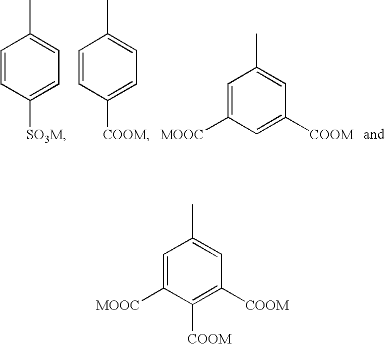

groups improve water dispersibility.

The known ink jet inks have the disadvantage of badly bleeding when they are printed onto paper.

### DESCRIPTION OF THE INVENTION

It is an object of the invention to provide an ink jet ink which exhibits good intercolour bleeding control on printing.

This object is achieved by an ink jet ink comprising a modified pigment, characterized in that the modified pigment is a pigment having attached organic groups of substituted or unsubstituted 1,2-benzenedicarboxylic acids and/or salts thereof.

The attached organic groups of substituted or unsubstituted 1,2-benzenedicarboxylic acids and/or salts thereof can be attached to the pigment via a carbon atom of the benzene ring.

The attached organic groups of substituted or unsubstituted 1,2-benzenedicarboxylic acid groups and/or salts thereof can be compounds of the general formula 1 or 2,

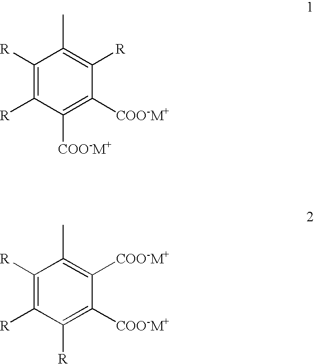

where
each R is the same or different and selected from H, acceptor groups, donor groups, alkyl or aryl groups having acceptor and donor groups respectively, hydrophilic and/or hydrophobic groups and M⁺ is selected from H⁺, Na⁺, K⁺, Li⁺, NH₄⁺, trimethylammonium (CH₃)₃NH⁺ and tetramethylammonium (CH₃)₄N⁺.

The R group is not a COO⁻M+group.

Acceptor groups can be —CO—R¹, —CN, —SO₂R¹ or —SO₂OR¹, where R¹=metal, H, alkyl, aryl, ammonium or functionalized alkyl or aryl. Donor groups can be SR², OR² or N(R²)₂, where R²=H, alkyl, aryl or functionalized alkyl or aryl. Hydrophilic groups can be SO₃Me (Me=metal) or —(CH₂—CH₂—O)ₙ—R², where n=1-45. Hydrophobic groups can be alkyl, aryl, fluoroalkyl, perfluoroalkyl, fluoroaryl or perfluoroaryl.

The organic groups R can be:

substituted or unsubstituted, branched or unbranched, an aliphatic group, for example radicals from alkanes, alkenes, alcohols, ethers, aldehydes, ketones, esters, hydrocarbons, sulphonic acids, amines, trialkylammonium salts, trialkylphosphonium salts or dialkylsulphonium salts,
a cyclic group, for example alicyclic hydrocarbons, for example cycloalkyls or cycloalkenyls, heterocyclic compounds, for example pyrrolidinyl, pyrrolinyl, piperidinyl or morpholinyl groups, aryl groups, for example phenyl, naphthyl or anthracenyl groups, or heteroaryl groups, for example imidazolyl, pyrazolyl, pyridinyl, thienyl, thiazolyl, furyl or indolyl groups,
a heterocyclic group comprising nitrogen or further heteroatoms and form a three-, four-, five-, six- or more-membered ring which is in turn substituted by H, alkyl or aryl groups having acceptor and donor substituents respectively or parts of cyclic systems having acceptor and donor substituents respectively and/or hydrophilic/hydrophobic groups,
substituted by further functional groups,
a chromophoric group or a dye, or
suitable reactive compounds, for example triarylammonium, triarylphosphonium, diarylsulphonium and aryliodonium salts.

The modified pigment can have the following structures:

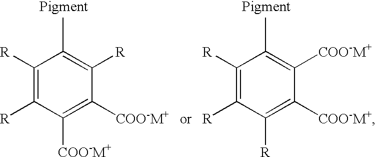

where one or more attached organic groups of substituted or unsubstituted 1,2-benzenedicarboxylic acid groups and/or salts thereof can be attached to the pigment.

The group of organic compounds of the general formula 1 and 2 can be custom tailored to the potential fields of use, since the principle of reaction permits for example not only the introduction of hydrophilic groups but also the introduction of lipophilic groups. The groups can also be ionic, polymeric or further reactive. The groups can be used to modify different, technologically useful properties of the pigment in a specific manner. For instance, the hydrophilicity of the carbon material can be increased to such an extent that the carbon material forms stable dispersions in aqueous media without use of a wetting agent.

Compounds of the general formula 1 and 2 can be for example:

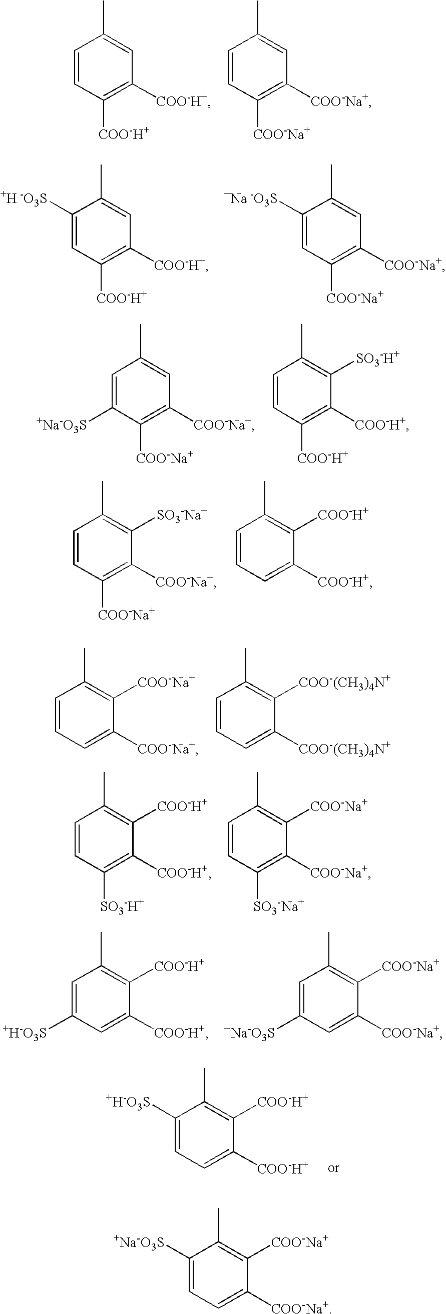

The modified pigments having, attached to the pigment, organic groups of substituted or unsubstituted 1,2-benzenedicarboxylic acids and/or salts thereof, may be obtainable by compounds of the general formula 3 and/or 4

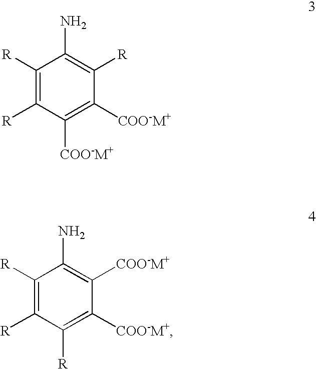

where R and M⁺ are each as defined above, being diazotized with alkali metal nitrite, preferably NaNO₂, in an acidic pH range and reacted with the pigment.

The modified pigments are obtainable by diazotization of compounds of the general formula 3 and/or 4

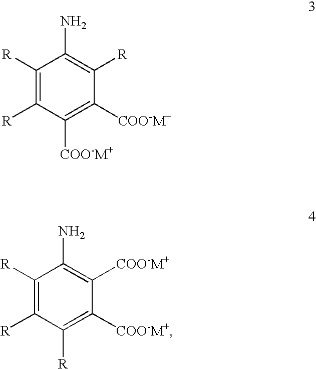

where R and M⁺ are each as defined above, with alkali metal nitrite, preferably NaNO₂, in an acidic pH range and reaction with the pigment.

One way of carrying out the process is first to mix the pigment and the compounds of the general formula 3 and/or 4, for example as an aqueous suspension, then to set an acidic pH range and subsequently to add the alkali metal nitrite.

One way of carrying out the process is to first mix the pigment and the compounds of the general formula 3 and/or 4 which are adjusted to an acidic pH range and then to add the alkali metal nitrite.

One way of carrying out the process is first to mix the compounds of the general formula 3 and/or 4 which have been adjusted to an acidic pH range, with alkali metal nitrite, and then to react the reaction product with the pigment.

Compounds of the general formula 3 and 4 can be for example:

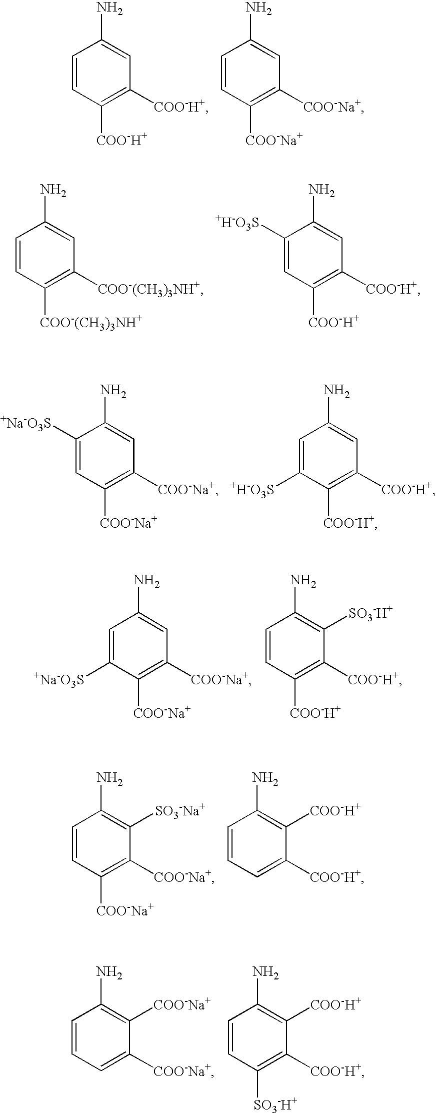

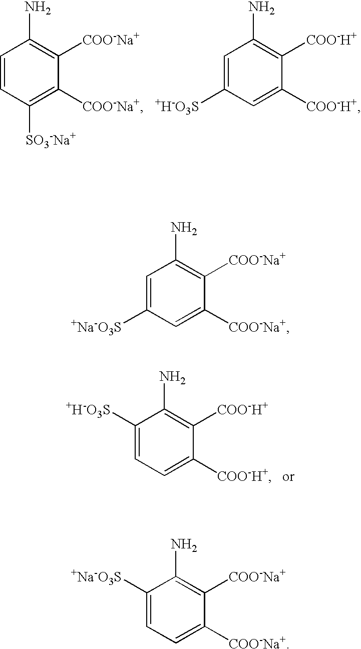

The pigment used can be carbon material or colour pigment. Colour pigment used can be for example yellow, orange, red, magenta, violet, blue, cyan, green or brown pigment. Colour pigment used can preferably be inorganic blue pigment, for example iron blue, ultramarine blue, cobalt blue or mixed phase blue pigment, or organic blue pigment, for example phthalocyanine blue or Indanthren blue.

Carbon material used can be carbon black, graphite powder, graphite fibres, carbon fibres, carbon fibrils, carbon nanotubes, aerogels, carbon fabrics, glassy carbon products, activated carbon and fullerenes.

Carbon black used can be furnace black, gas black, channel black, flame black, thermal black, acetylene black, plasma black, inversion black known from DE 195 21 565, Si-containing blacks known from WO 98/45361 or DE 196 13 796, or metal-containing blacks known from WO 98/42778, arc black and blacks which are by-products of chemical manufacturing operations.

The modified pigment according to the invention and/or the pigment can be activated by preceding reactions. These can be oxidation reactions for example. Useful oxidizing agents include for example ammonium peroxodisulphate, hydrogen peroxide, ozone, oxygen (pure or as air), potassium bromate and/or sodium perborate. The pigment can be an oxidized gas black.

It is possible to use carbon materials which are used as reinforcing filler in rubber mixtures. Pigment grade carbon blacks can be used. Further carbon materials can be: conductivity black, carbon material for UV stabilization, carbon material as filler in systems other than rubber, for example in bitumen or plastic, or carbon material useful as reducing agent in metallurgy.

The process for producing the modified pigments can be carried out in aqueous solution or aqueous suspension.

The pigment can be dispersed. The pigment dispersion can consist of the pigment, water and a wetting agent.

Useful wetting agents include anionic, cationic and/or nonionic wetting agents.

Anionic wetting agent used can be Tamol, Vandispers CB (lignosulphonate).

Cationic wetting agent used can be Akypoquat 132 (cationic fatty ester (CTFA: Lauroyl PG-Trimonium Chloride)) from Kao Chemicals GmbH, Bayowet FT 738 VP AC 2023 (quaternary fluoroalkylammonium iodide) from Bayer AG, DP2-7949 (aqueous solution of cationic homopolymers) from Ciba Geigy Chemicals, DP7-7961 (aqueous solution of cationic polymers) from Ciba Geigy Chemicals, DP7-7962 (aqueous solution of cationic polymers) from Ciba Geigy Chemicals, DP7-7963 (aqeuous solution of cationic polymers) from Ciba Geigy Chemicals, Epikuron 200 (phosphatidylcholine) from Lukas Meyer, Ethoxamine SF 11 (ethoxylated fatty amine with 11 mol of ethylene oxide) from Witco, Ethoxamine SF 15 (ethoxylated fatty amine with 15 mol of ethylene oxide) from Witco, Forbest 13 (compound neutr., acidic polyester and fatty alcohol) from Lukas Meyer, Forbest 610 (carboxylic acid-diamine preparation) from Lukas Meyer, Magnafloc 1797 (aqueous solution of cationic cross-linked condensation resins) from Ciba Speciality Chemicals, Protectol KLC 50 (dimethyl-C 12/14-alkylbenzylammonium chloride in water (about 50%)) from BASF, Rewoquat CPEM (cocopentaethoxymethylammonium methosulphate) from Witco Surfactants GmbH, Rewoquat RTM 50 (ricinoleic acid propylamido trimethylammonium methosulphate) from Witco Surfactants GmbH, Sochamine 35 (alkylimidazoline) from Witco Surfactants GmbH.

Nonionic wetting agent used can be a compound from the group consisting of crosslinked polyoxyethyleneacrylic acid, fatty alcohol oxethylates, nonylphenol polyglycol ethers, polyvinylpyrrolidone, glycerol fatty acid esters, propylene glycol fatty acid esters, sorbitan fatty acid esters, polyoxyethylene sorbitan fatty acid esters, tetraolein acid polyoxyethylene sorbitol, polyoxyethylene alkyl ethers, polyoxyethylene alkyl phenyl ethers, polyoxyethylene polyoxypropylene glycol, polyoxyethylene polyoxypropylene alkyl ethers, polyethylene glycol fatty acid esters, higher fatty acid alcohol esters, polyhydric alcohol fatty acid esters.

The pigment dispersion can be prepared by dispersing the pigment if appropriate together with the wetting agent or agents in water and if appropriate using bead mills, ultrasonicators, wing stirrers, dissolvers or an Ultra-Turrax for the dispersing. After dispersing, the carbon black dispersion can be centrifuged or filtered.

The acidic pH of the aqueous solution or suspension of the primary amine can be set using inorganic acids, preferably hydrochloric acid. The pH can be less than 6, preferably less than 3.

The pigment dispersion and the acidic aqueous solution of the primary amine can be mixed by stirring.

The sodium nitrite can be dissolved in water. The sodium nitrite solution can be added dropwise to the pigment dispersion/amine mixture. The reaction with sodium nitrite solution can be carried out at temperatures ranging from −5° C. to 30° C.

The modified pigments can be used as filler, reinforcing filler, UV stabilizer, conductivity carbon black or pigment.

The modified pigments according to the invention can be used in rubber, plastic, printing inks, liquid inks, ink jet inks, xerographic toners, varnishes and paints, bitumen, concrete and other building materials or paper. The modified pigments according to the invention can further be used as reducing agents in metallurgy. The modified pigments according to the invention can be used in the manufacture of rubber mixtures, for example in the manufacture of tyres.

The ink jet ink according to the invention may further comprise one or more additives, such as biocides, wetting agents, ketones, glycols, alcohols or mixtures thereof. These additives can be added for specific uses and for example also consist of monomeric, oligomeric or polymeric compounds. These additives can effect improvements of properties, such as degree of dispersion, storage stability, freeze stability, drying behaviour, filming behaviour, wettability and/or attachment to certain carrier materials, such as paper, metal, glass, polymers, fibres, leather, wood, concrete or rubber.

The biocide can be added in amounts of 0.01-1.0% by weight. Useful biocides include isothiazolinone derivatives, formaldehyde donors or combination products of the two classes of product. The biocide used can be for example Parmetol from Schülke & Mayr, Ebotec from Bode Chemie, Acticide from Thor Chemie or Proxel from Zeneca.

The ink jet ink according to the invention may comprise a dye.

The ink jet ink of the invention may comprise a dispersion-augmenting additive. The dispersion-augmenting additive can be used in amounts of 0.1-30% by weight, preferably 0.3-10% by weight, based on the ink. The molecular weight of the dispersion-augmenting additive can be 1000 to 20 000 g/mol, preferably 14 500 to 17 000 g/mol. The acid value of the dispersion-augmenting additive can be 120 to 320, preferably 180 to 280. Styrene-acrylic acid copolymers can be used as dispersion-augmenting additive. The copolymers can be random, alternating, block or graft copolymers. Joncryl 678, Joncryl 680, Joncryl 682 or Joncryl 690 from Johnson Polymer B.V. can be used as dispersion-augmenting additive for example. Fully ammonium- or alkali metal hydroxide-neutralized forms, especially NaOH-neutralized forms, of the styrene-acrylic acid copolymers can be used as dispersion-augmenting additive.

The ink jet ink of the invention can be used for printing textiles, foil, film, sheet and paper.

The present invention further provides a process for improving intercolour bleeding control of ink jet inks, the process being characterized in that the ink comprises a modified pigment, the modified pigment being a pigment having attached organic groups of substituted or unsubstituted benzenedicarboxylic acids and/or salts thereof. The benzenedicarboxylic acid may preferably be 1,2-benzenedicarboxylic acid.

The modified pigments having, attached to the pigment, organic groups of substituted or unsubstituted benzenedicarboxylic acids and/or salts thereof can be used for improving intercolour bleeding control of ink jet inks. The benzenedicarboxylic acid may preferably be 1,2-benzene-dicarboxylic acid.

The ink jet ink of the invention has the advantage that intercolour bleeding control is improved compared with the prior art.

### EXAMPLES

Carbon black S 160 is used as pigment in the examples. Gas black S 160 is a commercial product from Evonik Degussa GmbH.

### Example 1

### Preparation of Modified Carbon Blacks

This example describes the preparation of a carbon black modified with 0.8 mmol/g of the corresponding modifying agent.

70 g of carbon black S 160 and 800 g of ice-cooled distilled water are initially charged to a 2 l glass beaker and stirred with a ZR 2051 Heidolph stirrer having wing stirrers.

4.2 g of sodium nitrite are suspended in 8 g of ice and 12 g of distilled water in a 100 ml glass beaker using a magnetic stirrer.

The Table 1 amounts of modifying agent are each slurried up in a 40 ml glass beaker in 45 g of ice-cold distilled water and admixed with 16.3 g of concentrated hydrochloric acid with cooling. Then, the sodium nitrite solution is carefully added.

This solution is then added dropwise during 30 minutes to the carbon black slurry with moderate stirring. Stirring is subsequently continued for an hour and the temperature of the reaction mixture is raised to 30° C. with the aid of an IKA Combimag RET magnetic stirrer/hotplate, and stirring is subsequently continued at 30° C. for 3 hours.

The reaction mixture is suction filtered through a blue ribbon filter and the filter cake is washed with 5 litres of distilled water. The filter cake is dried at 120° C. overnight.

TABLE 1

| | | Amount of |
|---|---|---|
| | | modifying agent |
| | Modifying agent | (g) |
| Modified | 6-Amino-2-naphthol- | 10.48 |
| carbon black 1 | carboxylic acid | |
| Modified | 4-Aminophthalic | 10.14 |
| carbon black 2 | acid | |
| Modified | 5-Aminoiso- | 10.14 |
| carbon black 3 | terephthalic acid | |
| Modified | 2-Aminoterephthalic | 10.14 |
| carbon black 4 | acid | |
| Modified | Sulphanilic acid | 9.70 |
| carbon black 5 | | |
| Modified | 5-Amino-2- | 13.40 |
| carbon black 6 | naphthalene- | |
| | sulphonic acid | |

### Example 2

### Preparation of Dispersions with the Modified Carbon Blacks

This example describes the preparation of a dispersion (Table 2) with the modified carbon blacks described under Example 1 and also with a Degussa carbon black S 160.

In each case, 60 g of carbon black and 340 g of distilled water are initially charged to a 1 litre stirred vessel and stirred in with a spatula. A pH between 8-9 is set with 2-(dimethylamino)ethanol. The slurry is predispersed using an Ultra-Turrax at 10 000 rpm with cooling. This predispersion is pumped with cooling through a Hiel ultrasonic flow cell with UIP 500 ultrasonicator in 3 passes using an MV-Z Ismatec gear pump at pump setting 27 and dispersed at maximum ultrasonicator power.

The use of carbon black S 160 leads to immediate thickening. A liquid dispersion 7 is unobtainable.

TABLE 2

| Dispersion | comprising: |
|---|---|
| Dispersion 1 | Modified carbon black 1 |
| Dispersion 2 | Modified carbon black 2 |
| Dispersion 3 | Modified carbon black 3 |
| Dispersion 4 | Modified carbon black 4 |
| Dispersion 5 | Modified carbon black 5 |
| Dispersion 6 | Modified carbon black 6 |
| Dispersion 7 | Carbon black S 160 |

### Example 3

### Preparation of Ink

Inks (Table 3) having a pigment content of 5% of the modified carbon black are formulated using the dispersions mentioned in Example 2. The inks further comprise 6% by weight of 2-pyrrolidone, 3% by weight of 1,5-pentanediol, 1.5% by weight of 2-propanol, 1.5% by weight of Liponic EG-07, 3% by weight of triethylene glycol dimethyl ether; made up to 100% by weight with distilled water.

The pH is set between 8-9 with 2-(dimethylamino)ethanol.

TABLE 3

| Ink | comprising: |
|---|---|
| Ink 1 | Dispersion 1 |
| Ink 2 | Dispersion 2 |
| Ink 3 | Dispersion 3 |
| Ink 4 | Dispersion 4 |
| Ink 5 | Dispersion 5 |
| Ink 6 | Dispersion 6 |

### Example 4

### Preparation of Ink 8

An ink 8 is formulated using a Cab-O-Jet 300 dispersion from Cabot. Cab-O-Jet consists of 15% by weight of a surface-functional carboxylated carbon black. The ink comprises 5% by weight of carbon black, 6% by weight of 2-pyrrolidone, 3% by weight of 1,5-pentanediol, 1.5% by weight of 2-propanol, 1.5% by weight of Liponic EG-07, 3% by weight of triethylene glycol dimethyl ether; made up to 100% by weight with distilled water.

The pH is set between 8-9 with 2-(dimethylamino)ethanol.

### Example 5

### Evaluation of Inks

Evaluation is done using the black bar, subdivided into 4 sizes, in the first 4 square colour blocks starting from left white, cyan, magenta and yellow from picture 2: graphic block for “bleeding” and “feathering” of the DIN model sample 33871-1:2003-10.

a. Visual Evaluation of Inks Comprising Modified Carbon Blacks and of Canon Original Ink

A ranking is established for bleeding ranging from 1=no bleeding, 2=very little bleeding, 3=little bleeding, 4=bleeding, 5=considerable bleeding, 6=very considerable bleeding.

The dicarboxylic acid in ortho position exhibits the least bleeding (Table 4).

TABLE 4

| Ink used | Bleeding |
|---|---|
| Ink 1 | 5 |
| Ink 2 | 1 to 2 |
| Ink 3 | 2 to 3 |
| Ink 4 | 2 to 3 |
| Ink 5 | 5 |
| Ink 6 | 4 to 5 |
| Ink 8 | 2 to 3 |
| Original Canon BCI-3eBK | 1 to 2 |
| ink | |

b. Bleed Measurement of Inks Comprising Modified Carbon Blacks and of Canon Original Ink

The measurement is done using a “Personal IAS” image analyzer from qea. In each case, the third widest of the black bar, subdivided into 4 widths, in the first 4 square colour blocks, starting from left white, cyan, magenta and yellow from picture 2: graphic block for “bleeding” and “feathering” of DIN model sample 33871-1:2003-10 is optically sensed and the average width measured in μm. Then, the value of the black bar in the white measuring square is deduced from the measured values of the black bar in the cyan, magenta and yellow measuring square. The resulting value is indicative of the extent of bleeding. The smaller the value, the less the bleeding.

The ortho-dicarboxylic acid here shows the least bleeding (Tables 5+6).

TABLE 5
Kompass Copy Office paper

| | Cyan | Magenta | Yellow |
|---|---|---|---|
| | width | width | width |
| | bleeding | bleeding | bleeding |
| Ink used | [μm] | [μm] | [μm] |
| Ink 2 | 6.2 | 0 | 2.9 |
| Ink 3 | 31.1 | 18.6 | 22.5 |
| Ink 4 | 18.1 | 11.4 | 26.9 |
| Ink 8 | 43.7 | 11.5 | 27.7 |
| Original Canon | 11.6 | 3.4 | 9.4 |
| BCI-3eBK ink | | | |

TABLE 6
Inapa Tecno Office paper

| | Cyan | Magenta | Yellow |
|---|---|---|---|
| | width | width | width |
| | bleeding | bleeding | bleeding |
| Ink used | [μm] | [μm] | [μm] |
| Ink 2 | 1 | 0.8 | 4.2 |
| Ink 3 | 43.5 | 10.4 | 49.6 |
| Ink 4 | 67.2 | 17.1 | 42.8 |
| Ink 8 | 72.8 | 26.2 | 42.2 |
| Original Canon | 20.2 | 7.7 | 15 |
| BCI-3eBK Ink | | | |

1-5. (canceled)

6. An ink jet ink comprising a pigment with attached organic groups, said organic groups comprising substituted or unsubstituted 1,2-benzenedicarboxylic acids and/or salts thereof.

7. The ink jet ink of claim 6, wherein said 1,2-benzenedicarboxylic acids are unsubsitituted and are attached to said pigment by a carbon atom of the benzene ring.

8. The ink jet ink of claim 6, wherein said substituted or unsubstituted 1,2-benzenedicarboxylic acids and/or salts thereof are compounds of formula 1 or 2 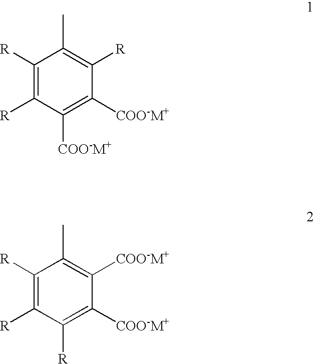
wherein:
each R is the same or different and is selected from the group consisting of: H, acceptor groups, donor groups, alkyl or aryl groups having acceptor and donor groups respectively, hydrophilic groups and hydrophobic groups; and
M⁺ is selected from H⁺, Na⁺, K⁺, Li⁺, NH₄⁺, (CH₃)₃NH⁺ and (CH₃)₄N⁺.

9. The ink jet ink of claim 8, wherein each R group is the same or different and is selected from the group consisting of: —CO—R¹, —CN, —SO₂R¹ and —SO₂R¹, where R¹ is a metal, H, alkyl, aryl, ammonium or functionalized alkyl or aryl.

10. The inkjet ink of claim 9, wherein R¹ is H.

11. The ink jet ink of claim 8, wherein each R group is the same or different and is selected from the group consisting of: SR², OR² and N(R²)₂, where R² is H, alkyl, aryl or a functionalized alkyl or aryl.

12. The ink jet ink of claim 11, wherein R² is H.

13. The ink jet ink of claim 8, wherein each R group is the same or different and is selected from the group consisting of: SO₃Me, wherein Me is a metal, and —(CH₂—CH₂—O)ₙ—R², where n=1-45 and R² is H, alkyl, aryl or a functionalized alkyl or aryl.

14. The ink jet ink of claim 6, wherein said pigment with attached organic groups comprises one of the following structures: 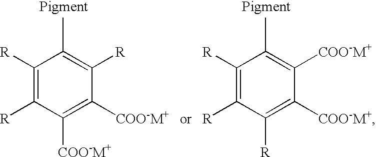
wherein:
each R is the same or different and is selected from the group consisting of: H, acceptor groups, donor groups, alkyl or aryl groups having acceptor and donor groups respectively, hydrophilic groups and hydrophobic groups; and
M⁺ is selected from H⁺, Na⁺, K⁺, Li⁺, NH₄⁺, (CH₃)₃NH⁺ and (CH₃)₄N⁺.

15. The ink jet ink of claim 6, wherein said substituted or unsubstituted 1,2-benzenedicarboxylic acids and/or salts thereof are selected from the group consisting of:

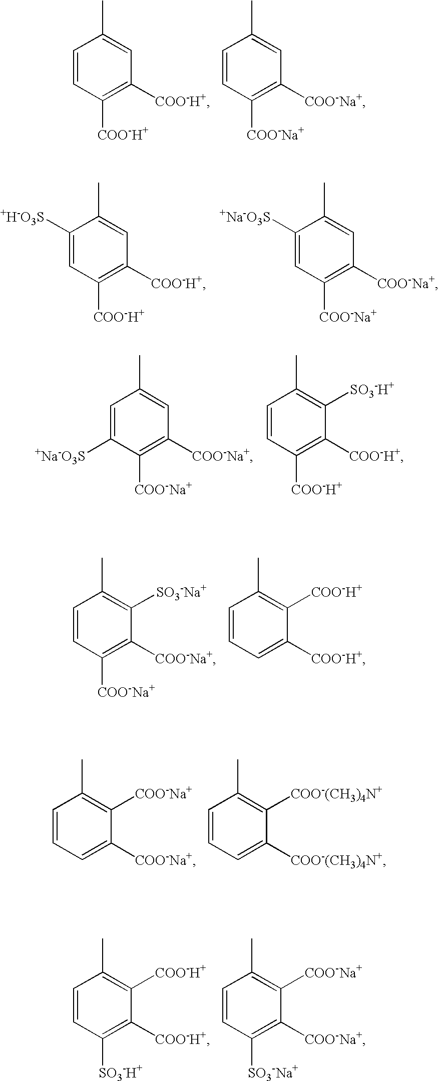

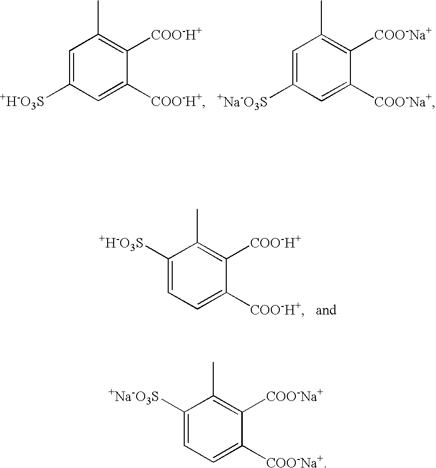

16. A process for improving intercolour bleeding control of ink jet inks, comprising modifying a pigment in said ink jet ink by attaching one or more organic groups of substituted or unsubstituted benzenedicarboxylic acids and/or salts thereof.

17. The process of claim 16, wherein said 1,2-benzenedicarboxylic acids are unsubsitituted and are attached to said pigment by a carbon atom of the benzene ring.

18. The process of claim 16, wherein said substituted or unsubstituted 1,2-benzenedicarboxylic acids and/or salts thereof are compounds of formula 1 or 2 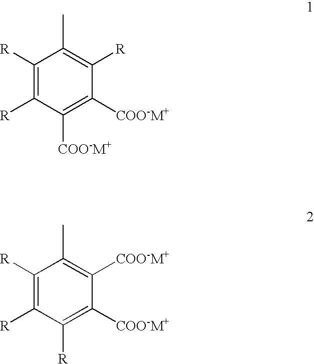
wherein:
each R is the same or different and is selected from the group consisting of: H, acceptor groups, donor groups, alkyl or aryl groups having acceptor and donor groups respectively, hydrophilic groups and hydrophobic groups; and
M⁺ is selected from H⁺, Na⁺, K⁺, Li⁺, NH₄⁺, (CH₃)₃NH⁺ and (CH₃)₄N⁺.

19. The process of claim 16, wherein said process results in the production of a modified pigment comprising one of the following structures: 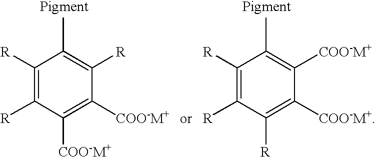
wherein:
each R is the same or different and is selected from the group consisting of: H, acceptor groups, donor groups, alkyl or aryl groups having acceptor and donor groups respectively, hydrophilic groups and hydrophobic groups; and
M⁺ is selected from H⁺, Na⁺, K⁺, Li⁺, NH₄⁺, (CH₃)₃NH⁺ and (CH₃)₄N⁺.

20. The process of claim 16, wherein said process results in the production of a modified pigment selected from the group consisting of:

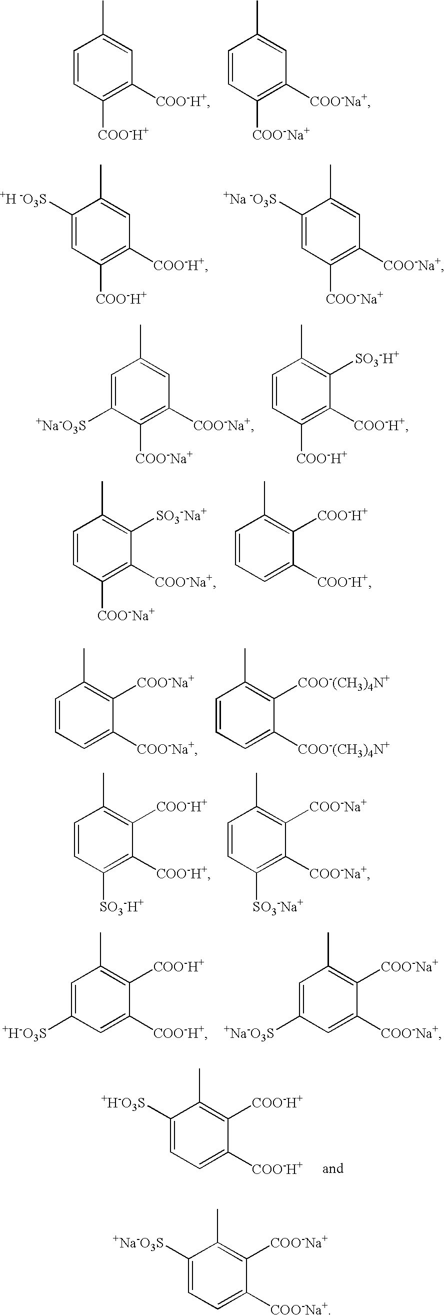

21. A textile, foil, film, sheet or paper printed with the ink jet ink of claim 6.

22. The textile, foil, film, sheet or paper of claim 21, wherein said ink jet ink comprises 1,2-benzenedicarboxylic acids that are unsubsitituted and that are attached to said pigment by a carbon atom of the benzene ring.

23. The textile, foil, film, sheet or paper of claim 22, wherein said ink jet ink comprises 1,2-benzenedicarboxylic acids that are compounds of formula 1 or 2 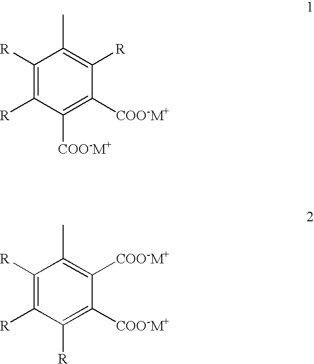
wherein:
each R is the same or different and is selected from the group consisting of: H, acceptor groups, donor groups, alkyl or aryl groups having acceptor and donor groups respectively, hydrophilic groups and hydrophobic groups; and
M⁺ is selected from H⁺, Na⁺, K⁺, Li⁺, NH₄⁺, (CH₃)₃NH⁺ and (CH₃)₄N⁺.

24. The textile, foil, film, sheet or paper of claim 22, wherein said ink jet ink comprises a modified pigment having one of the following structures: 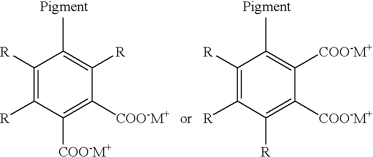
wherein:
each R is the same or different and is selected from the group consisting of: H, acceptor groups, donor groups, alkyl or aryl groups having acceptor and donor groups respectively, hydrophilic groups and hydrophobic groups; and
M⁺ is selected from H⁺, Na⁺, K⁺, Li⁺, NH₄⁺, (CH₃)₃NH⁺ and (CH₃)₄N⁺.

25. The textile, foil, film, sheet or paper of claim 22, wherein said ink jet ink comprises 1,2-benzenedicarboxylic acids selected from the group consisting of: [chemical structure]
